(19) United States
(12) Patent Application Publication
(10) Pub. No.: US 2023/0386608 A1
(43) Pub. Date: Nov. 30, 2023

(54) TARGETED CALLING OF OVERLAPPING COPY NUMBER VARIANTS

(71) Applicant: Illumina, Inc., San Diego, CA (US)

(72) Inventors: Xiao Chen, San Diego, CA (US); Michael A. Eberle, San Diego, CA (US)

(21) Appl. No.: 18/301,595

(22) Filed: Apr. 17, 2023

Related U.S. Application Data
(60) Provisional application No. 63/332,107, filed on Apr. 18, 2022.

(51) Int. Cl. G16B 30/10; G16B 20/10; G16B 20/20

(52) U.S. Cl. CPC G16B 30/10; G16B 20/10; G16B 20/20

(57) ABSTRACT

Disclosed herein include systems, devices, and methods for calling overlapping copy number variants (CNVs) of a gene. The gene can comprise a plurality of regions. The gene can have a plurality of CNVs. Two alleles of the gene of a subject can be determined based on a number of copies of each region of the plurality of regions and all CNVs of the plurality of CNVs of the gene comprising the region.

### CROSS-REFERENCE TO RELATED APPLICATIONS

This application claims the benefit of priority to U.S. Provisional Patent Application No. 63/332,107, filed Apr. 18, 2022. The content of this related application is incorporated herein by reference in its entirety for all purposes.

### BACKGROUND

### Field

This disclosure relates generally to the field of calling copy number variants, and more particularly to calling overlapping copy number variants.

### Background

In the population there exist common CNVs of a gene that overlap in positions. Due to the overlapping positions, a genome-wide CNV caller may make wrong calls when there is a mixture of signals from more than one CNV in a single sample. There is a need for a targeted method that calls the genotype of overlapping CNVs accurately.

### SUMMARY

Disclosed herein include methods of determining alleles of a gene (or genotyping a gene) of a subject. In some embodiments, a method for determining alleles of a gene of a subject is under control of a processor (e.g., a hardware processor) and comprises: receiving a plurality of sequence reads generated from a sample obtained from a subject. The method can comprise: aligning the plurality of sequence reads to a reference genome sequence to obtain a plurality of aligned sequence reads comprising sequence reads aligned to a gene in the reference genome sequence. The gene can comprise a plurality of regions. Two copy number variants (CNVs) of a plurality of CNVs (or variants) of the gene can each comprise one or more regions of the plurality of regions. The two CNVs can differ by at least one region of the plurality of regions. The method can include: determining a number of the sequence reads aligned to each region of the plurality of regions of the gene in the reference genome sequence. The method can include: determining a number of copies (or observed or estimated copies) of each region of the plurality of regions based on the number of the sequence reads aligned to the region. The method can include: determining two alleles of the gene of the subject based on the number of copies of each region of the plurality of regions and all CNVs (or each CNV) of the plurality of CNVs comprising the region. Each of the two alleles of the gene of the subject can comprise one or more regions of the plurality of regions.

In some embodiments, the plurality of regions comprises a plurality of consecutive and/or non-overlapping regions. A number of the plurality of regions can be 2 to 10. One, one or more, or each of the plurality of regions can be 1 kilobase (kb) to 100 kb in length. In some embodiments, a number of the plurality of CNVs is 2 to 10. In some embodiments, one CNV of the plurality of CNVs do not overlap with one or more other CNVs of the plurality of CNVs. Two or more of the plurality of CNVs do not overlap (or are non-overlapping CNVs). The two CNVs of the plurality of CNVs of the gene do not overlap (or are non-overlapping CNVs). No CNVs of the plurality of CNVs overlap (or all CNVs of the plurality of CNVs are non-overlapping CNVs). In some embodiments, one CNV of the plurality of CNVs overlaps with one or more other CNVs of the plurality of CNV. Two CNVs of the plurality of CNVs of the gene overlap (or are overlapping CNVs). The two CNVs of the plurality of CNVs of the gene overlap (or are overlapping CNVs). The two CNVs of the plurality of CNVs of the gene can comprise an identical region of the plurality of regions (or are overlapping CNVs). In some embodiments, each CNV of the plurality of CNVs of the gene comprises one or more regions of the plurality of regions. Each CNV of the plurality of CNVs can differ from every other CNV of the plurality of CNVs by at least one region of the plurality of regions.

In some embodiments, a first region, a second region, and a third region of the plurality of regions are consecutive and non-overlapping. A first CNV of the two CNVs can comprise the first region and the second region, not the third region. A second CNV of the two CNVs can comprise the second region and the third region, not the first region. In some embodiments, a first region, a second region, and a third region of the plurality of regions are consecutive and non-overlapping. A first CNV of the two CNVs can comprise the first region, the second region, and the third region. A second CNV of the two CNVs can comprise the second region, not the first region and the third region. Determining the two alleles of the gene of the subject can comprise: determining two alleles of the gene of the subject based on the number of copies of the first region and the number of copies of the second region, not the number of copies of the third region. The third region can be shorter or substantially shorter than the first region. In some embodiments, a first CNV and a second CNV of the plurality of CNVs comprise no common region.

In some embodiments, the number of the sequence reads aligned to each region of the plurality of regions of the gene comprises a raw number or a normalized and/or GC-corrected number of the sequence reads aligned to each region of the plurality of regions of the gene. In some embodiments, determining the number of copies of each region of the plurality of regions comprises: determining the number of copies of each region of the plurality of regions using the number of the sequence reads aligned to the region based on a normalized and/or GC-corrected number of the sequence reads aligned to each region of the plurality of regions of the gene in the reference genome sequence. The method can further comprise: determining the normalized number of the sequence reads aligned to each region of the plurality of regions of the gene in the reference genome sequence using (1a) a depth of the sequence reads aligned to each region of the plurality of regions of the gene in the reference genome sequence, (1b) a length of the region of the gene, (2a) a depth of sequence reads of the plurality of sequence reads aligned to each of a plurality of regions of the reference genome sequence other than a genetic locus comprising the gene, and/or (2b) a length of each of the plurality of regions of the reference genome sequence other than the genetic locus comprising the gene. The method can further comprise: determining the GC corrected number of the sequence reads aligned to each region of the plurality of regions of the gene in the reference genome sequence from the number or the normalized number of the sequence reads aligned the region of gene in the reference genome sequence using a GC content of the region of the gene in the reference genome sequence.

In some embodiments, the number of copies of each region comprises the number of copies of each region relative to a reference number of copies of the region. The reference number of copies of the region can be 2. In some embodiments, determining the number of copies of each region of the plurality of regions comprises: determining a difference in the number of copies of each region of the plurality of regions, relative to a reference number of copies of the region, based on the number of the sequence reads aligned to the region. Determining the two alleles of the gene of the subject can comprise: determining the two alleles of the gene of the subject using the difference in the number of copies of each region of the plurality of regions, relative to the reference number of copies of the region, and all CNVs of the plurality of CNVs comprising the region.

In some embodiments, a first allele of the two alleles comprises a duplication (e.g., having two copies) of a CNV of the plurality of CNVs. A first allele of the two alleles can comprise a deletion (e.g., having zero copy) of a CNV of the plurality of CNVs. In some embodiments, a first allele of the two alleles can comprise one copy of a CNV of the plurality of CNVs. In some embodiments, a second allele of the two alleles comprises a duplication (e.g., having two copies) of a CNV of the plurality of CNVs. A second allele of the two alleles can comprise a deletion (e.g., having zero copy) of a CNV of the plurality of CNVs. In some embodiments, a second allele of the two alleles comprises one copy of a CNV of the plurality of CNVs.

In some embodiments, determining the two alleles of the gene of the subject comprises: determining (i) a number of copies a first CNV in a first allele of the two alleles of the gene of the subject and (ii) a number of copies of a second CNV in a second allele of the two alleles of the gene of the subject such that (a) the number of copies of a region of the plurality of regions in the first CNV and not the second CNV is the number of copies of the first CNV, (b) the number of copies of a region of the plurality of regions in the first CNV and the second CNV is the sum of the number of copies of the first CNV and the number of copies of the second CNV, and/or (c) the number of copies of a region of the plurality of regions in the second CNV and not the first CNV is the number of copies of the second CNV.

In some embodiments, the plurality of CNVs is predetermined (or the plurality of CNVs is known). The plurality of regions can be predetermined. In some embodiments, the method further comprises: receiving the plurality of CNVs. The method can further comprise: determining the plurality of regions using the plurality of CNVs. Receiving the plurality of CNVs can comprise: determining the plurality of CNVs.

In some embodiments, the method further comprises: creating a file or a report representing or comprising alleles of the gene of the subject and/or the one or more regions of the plurality of regions in each of the two alleles. In some embodiments, the method further comprises: generating a user interface (UI) comprising a UI element representing or comprising alleles of the gene of the subject and/or the one or more regions of the plurality of regions in each of the two alleles.

In some embodiments, the plurality of sequence reads comprises sequence reads that are about 100 base pairs to about 1000 base pairs in length each. The plurality of sequence reads can comprise paired-end sequence reads and/or single-end sequence reads. The plurality of sequence reads is generated by whole genome sequencing (WGS), such as clinical WGS (cWGS). In some embodiments, the sample comprises cells, cell-free DNA, cell-free fetal DNA, amniotic fluid, a blood sample, a biopsy sample, or a combination thereof. The sample can be obtained directly from a subject. The sample can be generated from another sample obtained from a subject. The other sample can be obtained directly from the subject.

Disclosed herein include systems of determining alleles of a gene of a subject. In some embodiments, a system for determining alleles of a gene of a subject comprises: non-transitory memory configured to store executable instructions. The non-transitory memory can be configured to store a plurality of regions of a gene, and a plurality of copy number variants (CNVs) of the gene. Two CNVs of the plurality of CNVs of the gene each can comprise one or more regions of the plurality of regions and differ by at least one region of the plurality of regions. The system can comprise: a hardware processor in communication with the non-transitory memory. The hardware processor can be programmed by the executable instructions to perform: receiving a plurality of sequence reads generated from a sample obtained from a subject. The hardware processor can be programmed by the executable instructions to perform: aligning the plurality of sequence reads to a reference sequence to obtain a plurality of aligned sequence reads comprising sequence reads aligned to the gene in the reference genome sequence. The hardware processor can be programmed by the executable instructions to perform: determining a number of copies of each region of the plurality of regions based on a number of the sequence reads aligned to the region. The hardware processor can be programmed by the executable instructions to perform: determining two alleles of the gene of the subject, each comprising one or more regions of the plurality of regions, based on the number of copies of each region of the plurality of regions and all CNVs of the plurality of CNVs comprising the region. In some embodiments, the reference sequence comprises a reference genome sequence.

In some embodiments, the plurality of regions comprises a plurality of consecutive and/or non-overlapping regions. A number of the plurality of regions can be 2 to 10. One, one or more, or each of the plurality of regions can be 1 kilobase (kb) to 100 kb in length. In some embodiments, a number of the plurality of CNVs is 2 to 10. In some embodiments, one CNV of the plurality of CNVs do not overlap with one or more other CNVs of the plurality of CNVs. Two or more of the plurality of CNVs do not overlap (or are non-overlapping CNVs). The two CNVs of the plurality of CNVs of the gene do not overlap (or are non-overlapping CNVs). No CNVs of the plurality of CNVs overlap (or all CNVs of the plurality of CNVs are non-overlapping CNVs). In some embodiments, one CNV of the plurality of CNVs overlaps with one or more other CNVs of the plurality of CNV. Two CNVs of the plurality of CNVs of the gene overlap (or are overlapping CNVs). The two CNVs of the plurality of CNVs of the gene overlap (or are overlapping CNVs). The two CNVs of the plurality of CNVs of the gene can comprise an identical region of the plurality of regions (or are overlapping CNVs). In some embodiments, each CNV of the plurality of CNVs of the gene comprises one or more regions of the plurality of regions. Each CNV of the plurality of CNVs can differ from every other CNV of the plurality of CNVs by at least one region of the plurality of regions.

In some embodiments, a first region, a second region, and a third region of the plurality of regions are consecutive and non-overlapping. A first CNV of the two CNVs can comprise the first region and the second region, not the third region. A second CNV of the two CNVs can comprise the second region and the third region, not the first region. In some embodiments, a first region, a second region, and a third region of the plurality of regions are consecutive and non-overlapping. A first CNV of the two CNVs can comprise the first region, the second region, and the third region. A second CNV of the two CNVs can comprise the second region, not the first region and the third region. Determining the two alleles of the gene of the subject can comprise: determining two alleles of the gene of the subject based on the number of copies of the first region and the number of copies of the second region, not the number of copies of the third region. The third region can be shorter or substantially shorter than the first region. In some embodiments, a first CNV and a second CNV of the plurality of CNVs comprise no common region.

In some embodiments, the hardware processor is further programmed by the executable instructions to perform: determining the number of the sequence reads aligned to each region of the plurality of regions of the gene in the reference sequence. In some embodiments, the number of the sequence reads aligned to each region of the plurality of regions of the gene comprises a raw number or a normalized and/or GC-corrected number of the sequence reads aligned to each region of the plurality of regions of the gene. In some embodiments, determining the number of copies of each region of the plurality of regions comprises: determining the number of copies of each region of the plurality of regions using the number of the sequence reads aligned to the region based on a normalized and/or GC-corrected number of the sequence reads aligned to each region of the plurality of regions of the gene in the reference sequence. In some embodiments, the hardware processor is further programmed by the executable instructions to perform: determining the normalized number of the sequence reads aligned to each region of the plurality of regions of the gene in the reference sequence using (1a) a depth of the sequence reads aligned to each region of the plurality of regions of the gene in the reference sequence, (1b) a length of the region of the gene, (2a) a depth of sequence reads of the plurality of sequence reads aligned to each of a plurality of regions of the reference sequence other than a genetic locus comprising the gene, and (2b) a length of each of the plurality of regions of the reference sequence other than the genetic locus comprising the gene. The hardware processor can be further programmed by the executable instructions to perform: determining the GC corrected number of the sequence reads aligned to each region of the plurality of regions of the gene in the reference genome sequence from the number or the normalized number of the sequence reads aligned the region of gene in the reference genome sequence using a GC content of the region of the gene in the reference genome sequence.

In some embodiments, the number of copies of each region comprises the number of copies of each region relative to a reference number of copies of the region. The reference number of copies of the region can be 2. In some embodiments, determining the number of copies of each region of the plurality of regions comprises: determining a difference in the number of copies of each region of the plurality of regions, relative to a reference number of copies of the region, based on the number of the sequence reads aligned to the region. Determining the two alleles of the gene of the subject comprises: determining the two alleles of the gene of the subject using the difference in the number of copies of each region of the plurality of regions, relative to the reference number of copies of the region, and all CNVs of the plurality of CNVs comprising the region.

In some embodiments, a first allele of the two alleles comprises a duplication (e.g., having two copies) of a CNV of the plurality of CNVs. A first allele of the two alleles can comprise a deletion (e.g., having zero copy) of a CNV of the plurality of CNVs. In some embodiments, a first allele of the two alleles can comprise one copy of a CNV of the plurality of CNVs. In some embodiments, a second allele of the two alleles comprises a duplication (e.g., having two copies) of a CNV of the plurality of CNVs. A second allele of the two alleles can comprise a deletion (e.g., having zero copy) of a CNV of the plurality of CNVs. In some embodiments, a second allele of the two alleles comprises one copy of a CNV of the plurality of CNVs.

In some embodiments, determining the two alleles of the gene of the subject comprises: determining (i) a number of copies a first CNV in a first allele of the two alleles of the gene of the subject and (ii) a number of copies of a second CNV in a second allele of the two alleles of the gene of the subject such that (a) the number of copies of a region of the plurality of regions in the first CNV and not the second CNV is the number of copies of the first CNV, (b) the number of copies of a region of the plurality of regions in the first CNV and the second CNV is the sum of the number of copies of the first CNV and the number of copies of the second CNV, and/or (c) the number of copies of a region of the plurality of regions in the second CNV and not the first CNV is the number of copies of the second CNV.

In some embodiments, the plurality of CNVs is predetermined (or the plurality of CNVs is known). The plurality of regions can be predetermined. In some embodiments, the hardware processor is further programmed by the executable instructions to perform: receiving the plurality of CNVs. The hardware processor can be further programmed by the executable instructions to perform: determining the plurality of regions using the plurality of CNVs. Receiving the plurality of CNVs can comprise: determining the plurality of CNVs.

In some embodiments, the hardware processor is further programmed by the executable instructions to perform: creating a file or a report representing or comprising alleles of the gene of the subject and/or the one or more regions of the plurality of regions in each of the two alleles. The hardware processor can be further programmed by the executable instructions to perform: creating a file or a report and/or generating a user interface (UI) comprising a UI element representing or comprising alleles of the gene of the subject and/or the one or more regions of the plurality of regions in each of the two alleles.

In some embodiments, the plurality of sequence reads comprises sequence reads that are about 100 base pairs to about 1000 base pairs in length each. The plurality of sequence reads can comprise paired-end sequence reads and/or single-end sequence reads. The plurality of sequence reads is generated by whole genome sequencing (WGS), such as clinical WGS (cWGS). In some embodiments, the sample comprises cells, cell-free DNA, cell-free fetal DNA, amniotic fluid, a blood sample, a biopsy sample, or a combination thereof. The sample can be obtained directly from a subject. The sample can be generated from another sample obtained from a subject. The other sample can be obtained directly from the subject.

Also disclosed herein include a non-transitory computer-readable medium storing executable instructions, when executed by a system (e.g., a computing system), causes the system to perform any method or one or more steps of a method disclosed herein.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 shows that the breakpoints called by a genome-wide copy number variant (CNV) caller can vary

FIG. 2 show targeted copy number (CN) calling with a one-dimensional mixture of Gaussians with constrained means.

FIGS. 3A-3B illustrates an example of solving a complex region with overlapping CNVs using a targeted method described herein.

FIG. 4 illustrate another example of solving a complex region with overlapping CNVs using a targeted method described herein.

FIGS. 5A-5B illustrate a further example of solving a complex region with overlapping CNVs using a targeted method described herein.

FIG. 6 is a flow diagram showing an exemplary method of determining alleles of a gene with overlapping CNVs.

FIG. 7 is a block diagram of an illustrative computing system configured to implement determining alleles of a gene with overlapping CNVs.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

In the population there exist common CNVs of a gene that overlap in positions. Due to the overlapping positions, genome-wide CNV calling may be inaccurate, for example, when there is a mixture of signals from more than one CNV in a single sample. A targeted method that calls the genotype of overlapping CNVs accurately is described herein. The method can take advantage of a prior knowledge of some or all possible CNVs that could exist in a given region of a gene, such as the CNVs shown in Table 1. The method can comprise receiving a plurality of sequence reads generated from a sample obtained from a subject. The method can comprise aligning the plurality of sequence reads to a reference genome sequence to obtain a plurality of aligned sequence reads comprising sequence reads aligned to a gene in the reference genome sequence. The gene can comprise a plurality of regions. Two copy number variants (CNVs) of a plurality of CNVs (or variants) of the gene can each comprise one or more regions of the plurality of regions. The two CNVs can differ by at least one region of the plurality of regions. The method can comprise determining a number of the sequence reads aligned to each region of the plurality of regions of the gene in the reference genome sequence. The method can comprise determining a number of copies (or observed or estimated copies) of each region of the plurality of regions based on the number of the sequence reads aligned to the region. The method can comprise determining two alleles of the gene of the subject based on the number of copies of each region of the plurality of regions and all CNVs (or each CNV) of the plurality of CNVs comprising the region. Each of the two alleles of the gene of the subject can comprise one or more regions of the plurality of regions.

Disclosed herein include a system of determining alleles of a gene of a subject. In some embodiments, the system comprises non-transitory memory configured to store executable instructions. The non-transitory memory can be configured to store a plurality of regions of a gene, and a plurality of copy number variants (CNVs) of the gene. Two CNVs of the plurality of CNVs of the gene each can comprise one or more regions of the plurality of regions and differ by at least one region of the plurality of regions. The system can comprise a hardware processor in communication with the non-transitory memory. The hardware processor can be programmed by the executable instructions to perform receiving a plurality of sequence reads generated from a sample obtained from a subject. The hardware processor can be programmed by the executable instructions to perform aligning the plurality of sequence reads to a reference sequence to obtain a plurality of aligned sequence reads comprising sequence reads aligned to the gene in the reference genome sequence. The hardware processor can be programmed by the executable instructions to perform determining a number of copies of each region of the plurality of regions based on a number of the sequence reads aligned to the region. The hardware processor can be programmed by the executable instructions to perform: determining two alleles of the gene of the subject, each comprising one or more regions of the plurality of regions, based on the number of copies of each region of the plurality of regions and all CNVs of the plurality of CNVs comprising the region. In some embodiments, the reference sequence comprises a reference genome sequence.

### Targeted Calling of Overlapping CNVs

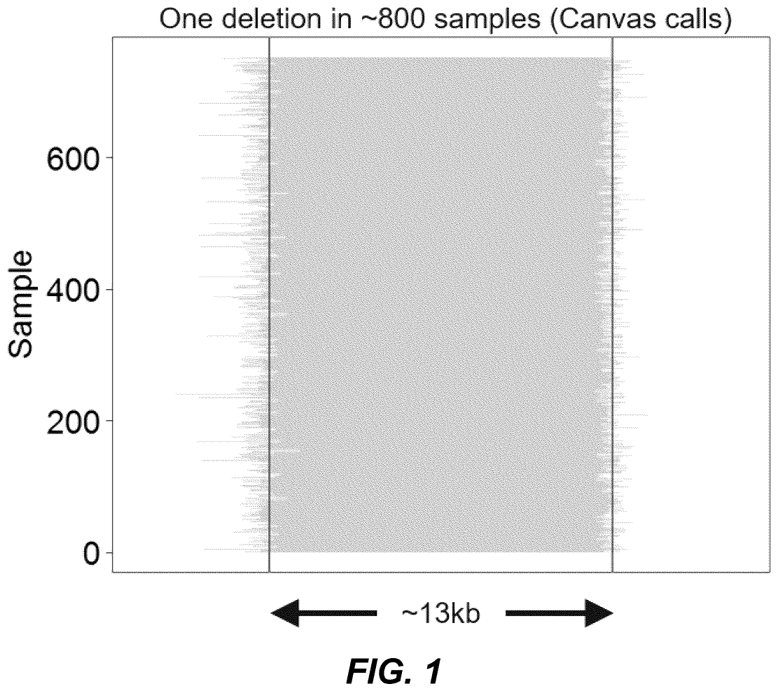

The majority of the copy number variants (CNVs) in an individual are common. Rediscovering the same variant in every sample using, for example, genome-wide CNV calling, can be very inefficient. Such genome-wide CNV calling can have low sensitivity and the resulting genotypes determined may be inaccurate. For example, it can be difficult to differentiate between (i) homozygous duplication where both alleles of a subject with two copies of a region of a gene) and (ii) one allele with no duplication and one allele with three copies of the region of the gene). Genome-wide CNV calling can be limited to large CNVs (e.g., 10 kb or longer). Breakpoints determined by genome-wide CNV calling can be highly variable (see FIG. 1 for an illustration) as the starting and ending positions may be called (or determined) incorrectly. Annotation can be tricky (or wrong). Targeted CNV calling can improve on all of these limitations of genome-wide CNV calling. In parallel targeted CNV calling can create benchmarking data to train single-individual genome-wide methods. Targeted CNV calling combined with targeted calling of other variant types can be used to genotype complicated but medically relevant regions of the genome.

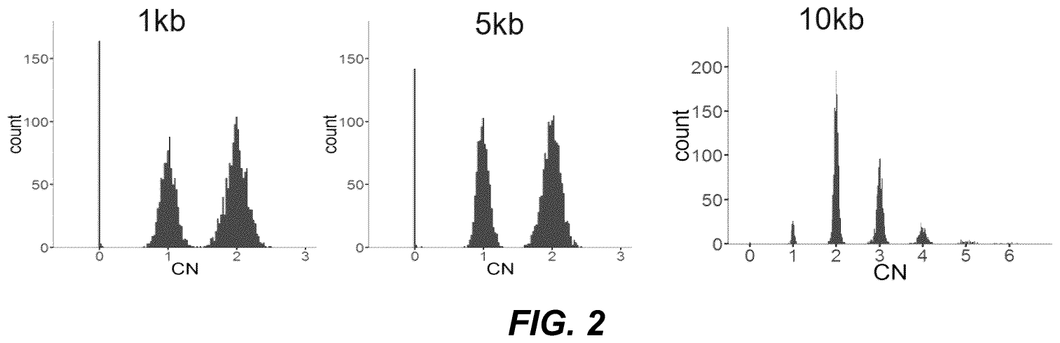

Targeted CNV calling can be performed using Gaussian mixture models of the population depth distribution. Use of Gaussian mixture models has been described in PCT Publication No. WO 2021/045947, entitled METHODS AND SYSTEMS FOR DIAGNOSING FROM WHOLE GENOME SEQUENCING DATA and U.S. Provisional Patent Application No. 63/197,936, entitled METHODS AND SYSTEMS FOR IDENTIFYING RECOMBINANT VARIANTS; the content of each of which is incorporated herein by reference in its entirety. Briefly, Gaussian mixture models can include a mixture of one-dimensional Gaussians with constrained means. The constrained means can be, for example, CN of 0, 1, 2, 3, 4, 5, 6, 7, 8, 9, and more. Use of such Gaussian mixture models can normalize out systemic biases and provide confidence in both variant and reference calls (CN equals 2). As a result, high sensitivity in small CNV regions (e.g., down to 1 kb; see FIG. 2 for an example) can be achieved. FIG. 2 show the performance of copy number calling with a one-dimensional mixture of Gaussians with constrained means and region lengths of 1 kilobase (kb) (the constrained means shown are CN of 1, 2, and 3), 5 kb (the constrained means shown are CN of 0, 1, 2, and 3), and 10 kb (the constrained means shown are CN of 0, 1, 2, 3, 4, 5, and 6). In FIG. 2, the y-axis (count) shows the number of samples, CN of 0 means homozygous deletion, and CN of 1 means deletion.

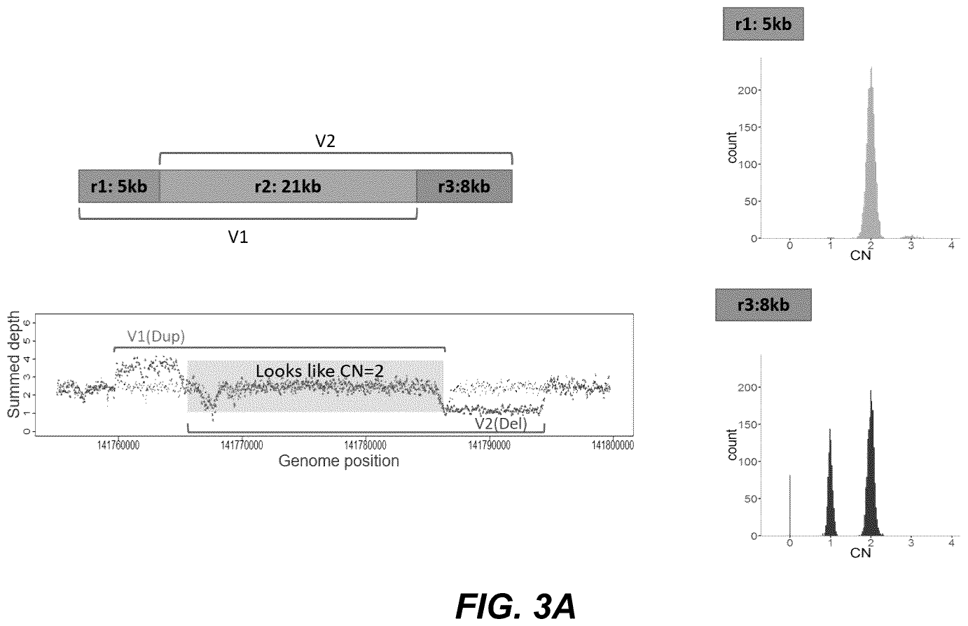

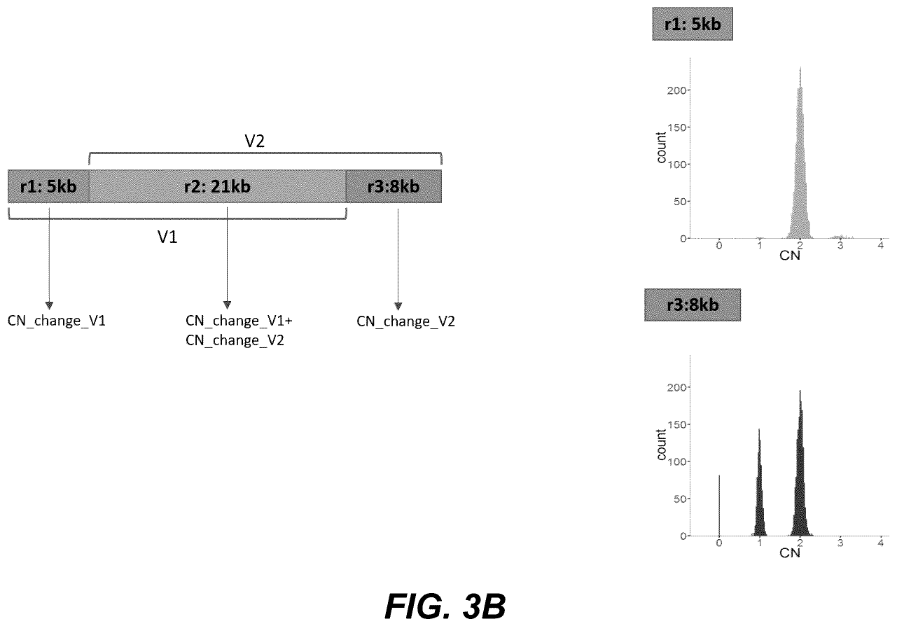

FIGS. 3A-3B illustrates an example of solving a complex region with overlapping CNVs using targeted CNV calling. Referring to FIG. 3A, a gene can have two overlapping variants (or a portion of the gene can have two overlapping variants). The gene (or a portion of the gene) can include three regions, a first region (labeled r1 in the figure), a second region (labeled r2 in the figure), and a third region (labeled r3 in the figure) as illustrated in FIG. 3A, top left panel. The three regions can be consecutive and non-overlapping as illustrated. The gene can have two CNVs (labeled V1 and V2 in the figure). A first CNV (labeled V1 in the figure) can include the first region (r1) and the second region (r2), not the third region (r3). A second CNV can include the second region (r2) and the third region (r3), not the first region (r1). The two CNVs both include the second region and are overlapping CNVs. As shown in FIG. 3A, top right panel, the first region (r1) can be duplicated in the population (as indicated by CN of 3). As shown in FIG. 3A, bottom right panel, the third region (r3) can be deleted in the population (as indicated by CN of 1 and 0). The CNs shown in FIG. 3A, top right panel and bottom right panel can be determined using a one-dimensional mixture of Gaussians with constrained means (the constrained means shown are CN of 0, 1, 2, 3, and 4). FIG. 3A bottom right panel shows the summed depth (or copy number) of the gene at various positions. Black dots in the figure show the summed depth (or coy number) of negative samples without any duplication or deletion. The grey dots in the figure show the summed depth (or copy number) of samples with first CNV (V1) duplication on one haplotype (or allele) and second CNV (V2) deletion on the other haplotype (or allele). A genome-wide CNV caller would have problem making the correct calls. For example, the caller may determine there is duplication (CN of 3) in the first region (r1), no duplication or deletion (CN of 2) in the second region (r2), and deletion (CN of 1) in the third region (r3). Since both regions are less than 10 kilobases in length, the difference in CN (CN of 3 or 1) from the CN of the reference (CN of 2) can be flattened by the genome-wide CNV caller.

Referring to FIG. 3B, with the prior knowledge that the gene (or a portion of the gene) can have two CNVs, the first CNV (V1) including the first region (r1) and the second region (r2), and the second CNV including the second region (r2) and the third region (r3), not the first region (r1), overlapping CNVs of this gene (or a portion thereof) can be determined. Since the first CNV (V1) includes the first region (r1) while the second CNV (V2) does not include the first region (r1), any observed CN change for the first region (r1) would be the CN change of the first CNV (“CN_change_V1” for the first region (r1) in the figure). Since the first CNV (V1) and the second CNV (V2) both include the second region (r2), any observed CN change for the second region (r2) would be the CN change of the sum of the CN change of the first CNV and the CN change of the second CNV (“CN_change_V1+CN_change_V2” for the second region (r2) in the figure). Since the first CNV (V1) does not include the third region (r3) while the second CNV (V2) includes the third region (r3), any observed CN change for the third region (r3) would be the CN change of the second CNV (“CN_change_V2” for the third region (r3) in the figure). The CN change of the first CNV and the CN change of the second CNV can be solved (or determined) that satisfies the observed summed depth (or CN), for example, of each of the regions shown in FIG. 3A, bottom left panel. The CN change of the first CNV (“CN_change_V1”) being positive one and the CN change of the second CNV (“CN_change_V2”) being negative one can satisfy the observed summed depth (or CN) or the observed change in summed depth (or CN), relative to a reference CN of two, of each of the three regions of a sample. The observed summed depth (or CN) can be determined based on the sequence reads aligned to each of the three regions. The sample can thus be determined to have one allele with V1 duplication and one allele with V2 deletion even though the summed depth (CN) appears as duplication of the first region (r1) and deletion of the third region (r3) in FIG. 3A, bottom left panel.

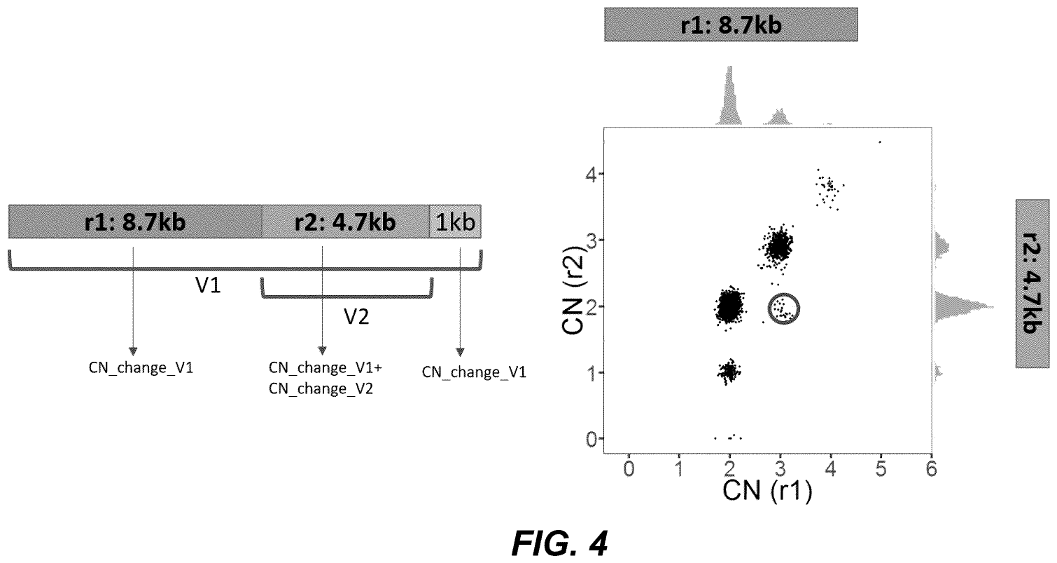

FIG. 4 illustrate another example of solving a complex region with overlapping CNVs using a targeted method described herein. The gene illustrated in FIG. 4, left panel can have two variants. The first variant (V1 in the figure) can have three regions, the first region (r1 in the figure), the second region (r2 in the figure), and the third region (the 1 kb region in the figure). The second variant (V2 in the figure) can have one region, the second region (r2), not the first region (r1) and the third region (the 1 kb region). Since the first CNV (V1) includes the first region (r1) while the second CNV (V2) does not include the first region (r1), any observed CN change for the first region (r1) would be the CN change of the first CNV (“CN_change_V1” for the first region (r1) in the figure). Since the first CNV (V1) and the second CNV (V2) both include the second region (r2), any observed CN change for the second region (r2) would be the CN change of the sum of the CN change of the first CNV and the CN change of the second CNV (“CN_change_V1+CN_change_V2” for the second region (r2) in the figure). Since the first CNV (V1) includes the third region (r3) while the second CNV (V2) does not include the third region (r3), any observed CN change for the third region (r3) would be the CN change of the first CNV (“CN_change_V1” for the third region (r3) in the figure). The CN change of the first CNV and the CN change of the second CNV can be solved (or determined) that satisfies the observed summed depth (or CN). In some embodiments, the CN change of the first CNV and the CN change of the second CNV can be solved (or determined) that satisfies the observed summed depth (or CN) of each of the regions. In some embodiments, the CN change of the first CNV and the CN change of the second CNV can be solved (or determined) that satisfies the observed summed depth (or CN) of the first region (r1) and the observed summed depth (or CN) of the second region (r2), not the observed summed depth (or CN) of the third region (r3). The observed summed depth (or CN) of the third region (r3) may not be used because the observed summed depth (or CN) of the first region (r1) and the observed summed depth (or CN) of the third region (r3) are identical and the third region (r3) is short. FIG. 4 shows that the third region (r3) is short relative to the length of the first region (r1) and in absolute term (1 kb).

FIG. 4, right panel shows the distribution of the combination of CN of the first region (r1) and the CN of the second region (r2) in samples. CN of the first region (r1) and the CN of the second region (r2) of a sample can be determined using a one-dimensional mixture of Gaussians with constrained means. Each dot in the figure represents a sample with a particular combination of the CN of the first region (r1) and the CN of the second region (r2). Each dot in the circle represents a sample with the CN change of the first region (r1) being positive one, relative to a reference CN of the first region (r1) of two; and the CN change of the second region (r2) being negative one, relative to a reference CN of the second region (r1) of one. The CN or the CN change a region can be determined based on the sequence reads aligned to each of the regions. The CN change of the first CNV (“CN_change_V1”) being positive one and the CN change of the second CNV (“CN_change_V2”) being negative one can satisfy the observed CN change of the first region (r1) being positive one and the CN change of the second region (r2) being negative one. The sample can thus be determined to have one allele with V1 duplication and one allele with V2 deletion. The third region (the 1 kb region) is short and its observed CN or CN change may not be considered in solving (or determining) the CN change of the first CNV (“CN_change_V1”) and the CN change of the second CNV (“CN_change_V2”) that can satisfy the observed CN change of the first region (r1) and the observed CN change of the second region (r2).

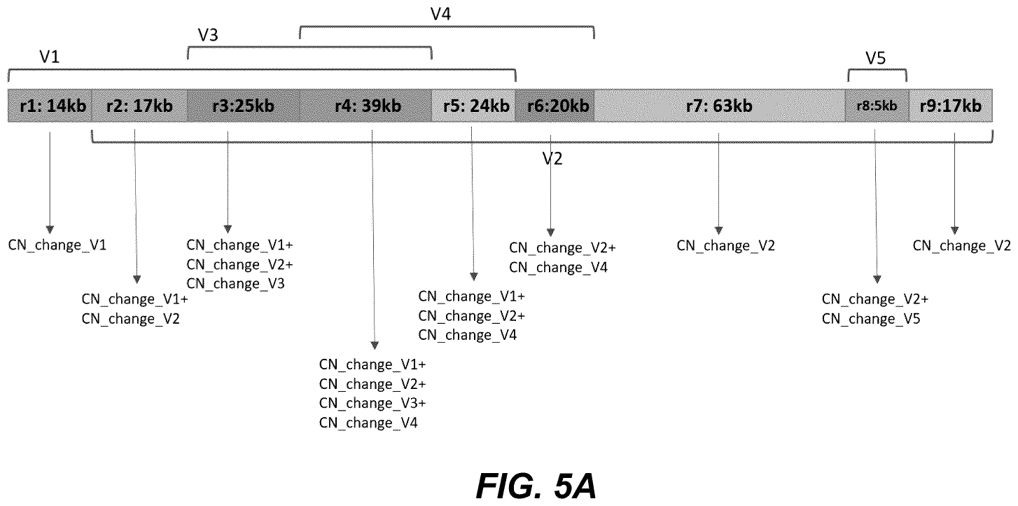

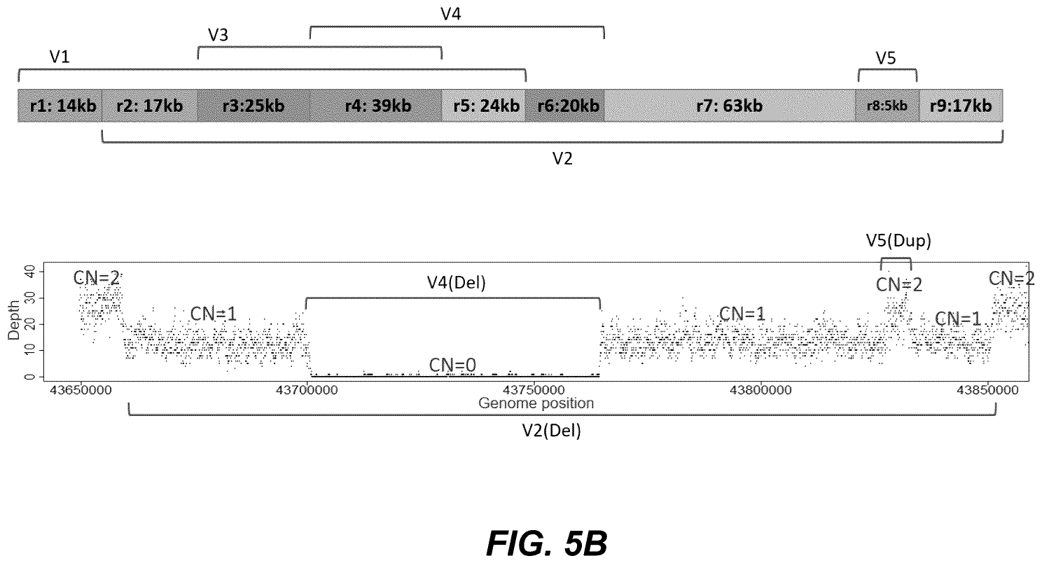

FIGS. 5A-5B illustrate a further example of solving a complex region with overlapping CNVs using a targeted method described herein. The gene (or a portion thereof) includes nine regions (r1 to r9). Some CNVs of the gene (or a portion thereof) illustrated in FIG. 5A are overlapping (these CNVs are overlapping CNVs). For example, the first CNV (V1 in the figure), the second CNV (V2 in the figure), the third CNV (V3 in the figure), and the fourth CNV (V4 in the figure) of the gene are overlapping. The first CNV (V1) and the fifth CNV (V5) are overlapping. Some CNVs of the gene (or a portion thereof) illustrated in FIG. 5A are non-overlapping (these CNVs are non-overlapping CNVs). For example, the first variant (V1) and the fifth variant (V5) are non-overlapping. The third variant (V3) and the fifth variant (V5) are non-overlapping. The fourth variant (V4) and the fifth variant (V5) are non-overlapping. Based on the various regions each CNV has, the CN change of each region can be determined. For example, the CN change of r1 is the CN change of the first variant (V1) as illustrated in the figure. The CN change of r4 is the sum of the CN change of the first CNV (V1), the CN change of the second CNV (V2), the CN change of the third CNV (V3), and the CN change of the fourth CNV (V4) as illustrated in the figure. The CN change of r9 is the CN change of the second CNV (V2) as illustrated in the figure.

Referring to FIG. 5B, bottom panel shows the observed CN change of a sample. The depth of the gene at various positions (or regions) correlate the CN of the gene at various positions. In the example shown in FIG. 5B, bottom panel, a depth of about 40 indicates the CN is 2, a depth of about 20 indicates the CN is 1, and a depth of about 0 indicates the CN is 0. The observed CN changes of the regions can be used to determine the CN changes of the CNVs using the relationship of the CN changes of the regions and the CN changes of the CNVs illustrated in FIG. 5A. The sample can be determined to have one allele with V2 deletion and another allele with V4 deletion and V5 duplication. For example, r1 has a CN of 2, which means the CN change of r1 (relative to a reference of two) is zero. Thus, the CN change of the first CNV (V1) is zero. R9 has a CN of one, which means the CN change of r2 (relative to a reference of two) is negative one. Thus CN change of the second CNV2 (v2) is negative one. r8 has a CN of 2, which means the CN change of r8 (relative to a reference of two) is zero. Since the observed CN change of r8 should be the sum of the CN change of the second CNV (V2) and the CN change of the fifth CNV (V5) and the CN change of the second CNV (V2) is negative one, the CN change of the fifth CNV (V5) is positive one. CN change of the third variant (V3) can be determined to be zero using the observed CN change of r3 being negative one; the observed CN change of r3 is the sum of the CN change of the first CNV (V1), the CN change of the second CNV (V2), and the CN change of the third CNV (V3); the CN change of the first CNV (V1) being zero, and the CN change of the second CNV (v2) being negative one. CN change of the fourth variant (V4) can be determined to be negative one using the observed CN change of r6 is negative two; the observed CN change of r6 is the sum of the CN change of the second CNV (V2) and the CN change of the fourth CNV (V4); and the CN change of the second CNV (V2) is negative one.

Table 1 shows exemplary copy number variants. The start and end positions of copy number variants can be used to determine the start and end positions of regions. Example 1 in Table 1 shows that two variants of a gene (or a portion thereof) can be at chr5:140842552-140859343 and chr5: 140834702-140848902 (the first CNV and the second CNV, respectively). Thus the gene (or a portion thereof) can have three regions, chr5:140842552-140834701 (140834702-1), 140834702-140848902, and 140848903 (140848902+1)-140859343 (the first region, the second region, and the third region, respectively). The observed CN change of the first region should be CN change of the first variant. The observed CN change of the second region should be the sum of CN change of the first variant and CN change of the second variant. The observed CN change of the third region should be the CN change of the first variant. Example 47 in Table 1 shows that three variants of a gene (or a portion thereof) can be at chr19:42749348-42862748, chr19: 42788173-43042773, and chr19: 42748348-42773348. Thus the gene (or a portion thereof) can have five regions, chr19: 42748348-42749347 (i.e., 42749348−1), 42749348-42773348, 42773349 (i.e., 42773348+1)-42788172 (i.e., 42788173-1), 42788173-42862748, 42862749 (i.e., 42862748+1)-43042773.

TABLE 1
Copy Number Variant Examples

| | Chr | Start | End | Variant_id | Regions |
|---|---|---|---|---|---|
| 1 | chr5 | 140842552 | 140859343 | c1-2163 | 140834702-140842551, |
| | chr5 | 140834702 | 140848902 | c1-216300 | 140842552-140848902, |
| | | | | | 140848903-140859343 |
| 2 | chr15 | 30483197 | 30493897 | c1-841 | 30483197-30489946, |
| | chr15 | 30489947 | 30495197 | c1-84100 | 30489947-30493897, |
| | | | | | 30493898-30495197 |
| 3 | chr3 | 162794345 | 162908547 | c1-1767 | 162794345-162807794, |
| | chr3 | 162807795 | 162827495 | c1-176700 | 162807795-162827495, |
| | | | | | 162827496-162908547 |
| 4 | chr6 | 22050711 | 22054117 | c1-2220 | 22050711-22052310, |
| | chr6 | 22052311 | 22053561 | c1-222000 | 22052311-22053561, |
| | | | | | 22053562-22054117 |
| 5 | chr16 | 70121338 | 70166738 | c2-191 | 70121338-70121487, |
| | chr16 | 70121488 | 70145588 | c2-19100 | 70121488-70145588, |
| | | | | | 70145589-70166738 |
| 6 | chr4 | 4107273 | 4157323 | c1-1817 | 4107273-4120522, |
| | chr4 | 4120523 | 4151073 | c1-181700 | 4120523-4151073, |
| | | | | | 4151074-4157323 |
| 7 | chr17 | 16752701 | 16845701 | c2-203 | 16752701-16805700, |
| | chr17 | 16805701 | 16845251 | c2-20300 | 16805701-16845251, |
| | | | | | 16845252-16845701 |
| 8 | chr15 | 32513499 | 32521249 | c4-165 | 32513249-32513498, |
| | chr15 | 32513249 | 32517499 | c4-16500 | 32513499-32517499, |
| | | | | | 32517500-32521249 |
| 9 | chr22 | 18137607 | 18147007 | c1-1579 | 18136483-18137606, |
| | chr22 | 18136483 | 18142783 | c2-304 | 18137607-18142783, |
| | | | | | 18142784-18147007 |
| 10 | chr11 | 54756309 | 54778113 | c1-362 | 54756309-54770496, |
| | chr11 | 54770497 | 54772221 | c1-363 | 54770497-54772221, |
| | | | | | 54772222-54778113 |
| 11 | chr12 | 126513271 | 126528771 | c1-597 | 126509689-126513270, |
| | chr12 | 126509689 | 126521289 | c1-596 | 126513271-126521289, |
| | | | | | 126521290-126528771 |
| 12 | chr11 | 55184051 | 55215401 | c1-368 | 55184051-55189383, |
| | chr11 | 55189384 | 55205413 | c1-369 | 55189384-55205413, |
| | | | | | 55205414-55215401 |
| 14 | chr1 | 159043410 | 159047860 | c5-15 | 159043410-159045009, |
| | chr1 | 159045010 | 159049110 | c1-119 | 159045010-159047860, |
| | | | | | 159047861-159049110 |
| 15 | chr11 | 63422993 | 63436393 | c2-73 | 63422993-63430456, |
| | chr11 | 63430457 | 63435157 | c1-379 | 63430457-63435157, |
| | | | | | 63435158-63436393 |
| 16 | chr12 | 11339411 | 11417911 | c1-474 | 11339411-11352665, |
| | chr12 | 11352666 | 11391766 | c5-59 | 11352666-11391766, |
| | | | | | 11391767-11417911 |
| 17 | chr22 | 42125709 | 42135305 | c1-1609 | 42125709-42129947, |
| | chr22 | 42129948 | 42140389 | c5-317 | 42129948-42135305, |
| | | | | | 42135306-42140389 |
| 18 | chr11 | 134727779 | 134747529 | c2-82 | 134727779-134732083, |
| | chr11 | 134732084 | 134737767 | c1-457 | 134732084-134737767, |
| | | | | | 134737768-134747529 |
| 19 | chr16 | 16617793 | 16619593 | c4-180 | 16611493-16617792, |
| | chr16 | 16611493 | 16621243 | c4-663 | 16617793-16619593, |
| | | | | | 16619594-16621243 |
| 20 | chr15 | 34510346 | 34518635 | c1-844 | 34415499-34510345, |
| | chr15 | 34415499 | 34527599 | c4-635 | 34510346-34518635, |
| | | | | | 34518636-34527599 |
| 21 | chr5 | 178682060 | 178686410 | c1-2194 | 178679826-178682059, |
| | chr5 | 178679826 | 178684838 | c1-2193 | 178682060-178684838, |
| | | | | | 178684839-178686410 |
| 22 | chr8 | 128750273 | 128753780 | c1-2698 | 128738986-128750272, |
| | chr8 | 128738986 | 128752584 | c1-2697 | 128750273-128752584, |
| | | | | | 128752585-128753780 |
| 23 | chr1 | 59582964 | 59583965 | c3-24 | 59581052-59582963, |
| | chr1 | 59581052 | 59584964 | c1-57 | 59582964-59583965, |
| | | | | | 59583966-59584964 |
| 24 | chr7 | 9595004 | 9596347 | c3-25 | 9593030-9595003, |
| | chr7 | 9593030 | 9597434 | c1-2406 | 9595004-9596347, |
| | | | | | 9596348-9597434 |
| 25 | chr5 | 110124958 | 110129658 | c1-2124 | 110124269-110124957, |
| | chr5 | 110124269 | 110139469 | c1-2123 | 110124958-110129658, |
| | | | | | 110129659-110139469 |
| 26 | chr7 | 142065528 | 142094542 | c1-2541 | 142060755-142065527, |
| | chr7 | 142060755 | 142087100 | c1-2540 | 142065528-142087100, |
| | | | | | 142087101-142094542 |
| 27 | chr9 | 61598112 | 61604812 | c4-1120 | 61598112-61598261, |
| | chr9 | 61598262 | 61648312 | c4-112800 | 61598262-61604812, |
| | | | | | 61604813-61648312 |
| 28 | chr7 | 76821432 | 76834310 | c2-446 | 76803133-76821431, |
| | chr7 | 76803133 | 76923683 | c5-530 | 76821432-76834310, |
| | | | | | 76834311-76923683 |
| 29 | chr1 | 196799066 | 196923990 | c1-148 | 196765870-196799065, |
| | chr1 | 196765870 | 196836720 | c4-1035 | 196799066-196836720, |
| | | | | | 196836721-196923990 |
| 30 | chr13 | 57178403 | 57214729 | c1-654 | 57178403-57212973, |
| | chr13 | 57212974 | 57214245 | c3-394 | 57212974-57214245, |
| | | | | | 57214246-57214729 |
| 31 | chr13 | 53660635 | 53666116 | c1-650 | 53660635-53665031, |
| | chr13 | 53665032 | 53667554 | c1-651 | 53665032-53666116, |
| | | | | | 53666117-53667554 |
| 32 | chr16 | 16236243 | 16245493 | c4-653 | 16235293-16236242, |
| | chr16 | 16235293 | 16270693 | c4-658 | 16236243-16245493, |
| | | | | | 16245494-16270693 |
| 33 | chr16 | 2608299 | 2615649 | c4-655 | 2608299-2608298, |
| | chr16 | 2608299 | 2685949 | c4-65500 | 2608299-2615649, |
| | | | | | 2615650-2685949 |
| 34 | chr15 | 24870556 | 24872357 | c1-830 | 24870556-24871842, |
| | chr15 | 24871843 | 24873586 | c1-831 | 24871843-24872357, |
| | | | | | 24872358-24873586 |
| 35 | chr20 | 1580354 | 1613054 | c1-1485 | 1572604-1580353, |
| | chr20 | 1572604 | 1605754 | c1-148500 | 1580354-1605754, |
| | | | | | 1605755-1613054 |
| 36 | chr19 | 37850681 | 37854823 | c1-1209 | 37850681-37852730, |
| | chr19 | 37852731 | 37854281 | c1-120900 | 37852731-37854281, |
| | | | | | 37854282-37854823 |
| 37 | chr9 | 133070640 | 133085790 | c2-505 | 133060780-133070639, |
| | chr9 | 133060780 | 133085790 | c2-50500 | 133070640-133085790, |
| | | | | | 133085791-133085790 |
| 38 | chr16 | 2646649 | 2650399 | c4-176 | 2636899-2646648, |
| | chr16 | 2636899 | 2685899 | c5-475 | 2646649-2650399, |
| | | | | | 2650400-2685899 |
| 39 | chr1 | 143673750 | 143680050 | c1-106 | 143541000-143673749, |
| | chr1 | 143541000 | 143708814 | c1-105 | 143673750-143680050, |
| | | | | | 143680051-143708814 |
| 40 | chr2 | 90225084 | 90228384 | c5-137 | 89868440-90225083, |
| | chr2 | 89868440 | 90265889 | c4-1062 | 90225084-90228384, |
| | | | | | 90228385-90265889 |
| 41 | chr6 | 31026229 | 31027303 | c3-217 | 31026229-31027083, |
| | chr6 | 31027084 | 31028944 | c1-2234 | 31027084-31027303, |
| | | | | | 31027304-31028944 |
| 42 | chr6 | 66330907 | 66333277 | c1-2278 | 66298835-66330906, |
| | chr6 | 66298835 | 66339023 | c1-2277 | 66330907-66333277, |
| | | | | | 66333278-66339023 |
| 43 | chr7 | 6097832 | 6099456 | c1-2400 | 6082319-6097831, |
| | chr7 | 6082319 | 6104169 | c1-2399 | 6097832-6099456, |
| | | | | | 6099457-6104169 |
| 44 | chr22 | 44169061 | 44170143 | c3-395 | 44168098-44169060, |
| | chr22 | 44168098 | 44172142 | c1-1612 | 44169061-44170143, |
| | | | | | 44170144-44172142 |
| 45 | chr5 | 20419583 | 20428365 | c1-2047 | 20419583-20420975, |
| | chr5 | 20420976 | 20438322 | c2-381 | 20420976-20428365, |
| | | | | | 20428366-20438322 |
| 46 | chr22 | 42522174 | 42575974 | c2-311 | 42505578-42522173, |
| | chr22 | 42505578 | 42554428 | c2-310 | 42522174-42554428, |
| | | | | | 42554429-42575974 |
| 47 | chr19 | 42749348 | 42862748 | c2-239 | 42748348-42749347, |
| | chr19 | 42788173 | 43042773 | c1-1217 | 42749348-42773348, |
| | chr19 | 42748348 | 42773348 | c2-23900 | 42773349-42788172, |
| | | | | | 42788173-42862748, |
| | | | | | 42862749-43042773 |
| 48 | chr15 | 24442772 | 24446822 | c2-166 | 24427094-24428211, |
| | chr15 | 24428212 | 24517862 | c1-829 | 24428212-24442771, |
| | chr15 | 24427094 | 24477444 | c1-828 | 24442772-24446822, |
| | | | | | 24446823-24477444, |
| | | | | | 24477445--24517862 |
| 49 | chr19 | 40843544 | 40875494 | c1-1214 | 40842595-40843543, |
| | chr19 | 40849774 | 40881524 | c1-1215 | 40843544-40847245, |
| | chr19 | 40842595 | 40847245 | c1-1213 | 40847246-40849773, |
| | | | | | 40849774-40875494, |
| | | | | | 40875495-40881524 |
| 50 | chr13 | 52252365 | 52337015 | c5-78 | 52252365-52306541, |
| | chr13 | 52306542 | 52327292 | c1-648 | 52306542-52327292, |
| | | | | | 52327293-52337015 |
| 51 | chr5 | 17619161 | 17620708 | c1-2044 | 17597991-17598990, |
| | chr5 | 17598991 | 17628641 | c4-1129 | 17598991-17610741, |
| | chr5 | 17618760 | 17645410 | c1-2043 | 17610742-17618759, |
| | chr5 | 17597991 | 17610741 | c4-112900 | 17618760-17619160, |
| | | | | | 17619161-17620708, |
| | | | | | 17620709-17628641, |
| | | | | | 17628642-17645410 |
| 52 | chr13 | 18754491 | 18773801 | c1-611 | 18754491-18764048, |
| | chr13 | 18764049 | 18783249 | c1-612 | 18764049-18767599, |
| | chr13 | 18767600 | 18786909 | c1-613 | 18767600-18773801, |
| | chr13 | 18785409 | 18801610 | c4-600 | 18773802-18783249, |
| | chr13 | 18795457 | 18802157 | c1-614 | 18783250-18785408, |
| | | | | | 18785409-18786909, |
| | | | | | 18786910-18795456, |
| | | | | | 18795457-18801610, |
| | | | | | 18801611-18802157 |
| 53 | chr4 | 9102877 | 9125277 | c1-1824 | 8955922-8971123, |
| | chr4 | 8975214 | 8998814 | c1-1823 | 8971124-8974872, |
| | chr4 | 8971124 | 9156224 | c4-1080 | 8974873-8975213, |
| | chr4 | 8955922 | 8974872 | c2-346 | 8975214-8987613, |
| | chr4 | 8987614 | 8998764 | c1-182300 | 8987614-8998814, |
| | | | | | 8998815-9102876, |
| | | | | | 9102877-9125277, |
| | | | | | 9125278-9156224, |
| | | | | | 9156225-8998764 |
| 54 | chr19 | 43172033 | 43236783 | c2-240 | 43141382-43155697, |
| | chr19 | 43196934 | 43260284 | c1-1220 | 43155698-43172032, |
| | chr19 | 43141382 | 43240682 | c1-1218 | 43172033-43196933, |
| | chr19 | 43155698 | 43346798 | c1-1219 | 43196934-43236783, |
| | chr19 | 43323674 | 43328874 | c2-241 | 43236784-43240682, |
| | | | | | 43240683-43260284, |
| | | | | | 43260285-43323673, |
| | | | | | 43323674-43328874, |
| | | | | | 43328875-43346798 |
| 55 | chr18 | 14275701 | 14295101 | c1-1089 | 14250504-14266361, |
| | chr18 | 14280018 | 14299518 | c1-1090 | 14266362-14270754, |
| | chr18 | 14285190 | 14304740 | c1-1091 | 14270755-14275700, |
| | chr18 | 14266362 | 14285812 | c1-1088 | 14275701-14280017, |
| | chr18 | 14250504 | 14270754 | c2-221 | 14280018-14285189, |
| | | | | | 14285190-14285812, |
| | | | | | 14285813-14295101, |
| | | | | | 14295102-14299518, |
| | | | | | 14299519-14304740, |
| 56 | chr21 | 13870937 | 13890292 | c3-448 | 13853803-13861097, |
| | chr21 | 13853803 | 13863003 | c2-296 | 13861098-13863003, |
| | chr21 | 13867898 | 13887398 | c1-1547 | 13863004-13867897, |
| | chr21 | 13861098 | 13880348 | c1-154700 | 13867898-13870936, |
| | chr21 | 13871026 | 13900282 | c2-297 | 13870937-13871025, |
| | | | | | 13871026-13880348, |
| | | | | | 13880349-13887398, |
| | | | | | 13887399-13890292, |
| | | | | | 13890293-13900282 |

Determining Alleles of a Gene with Overlapping CNVs

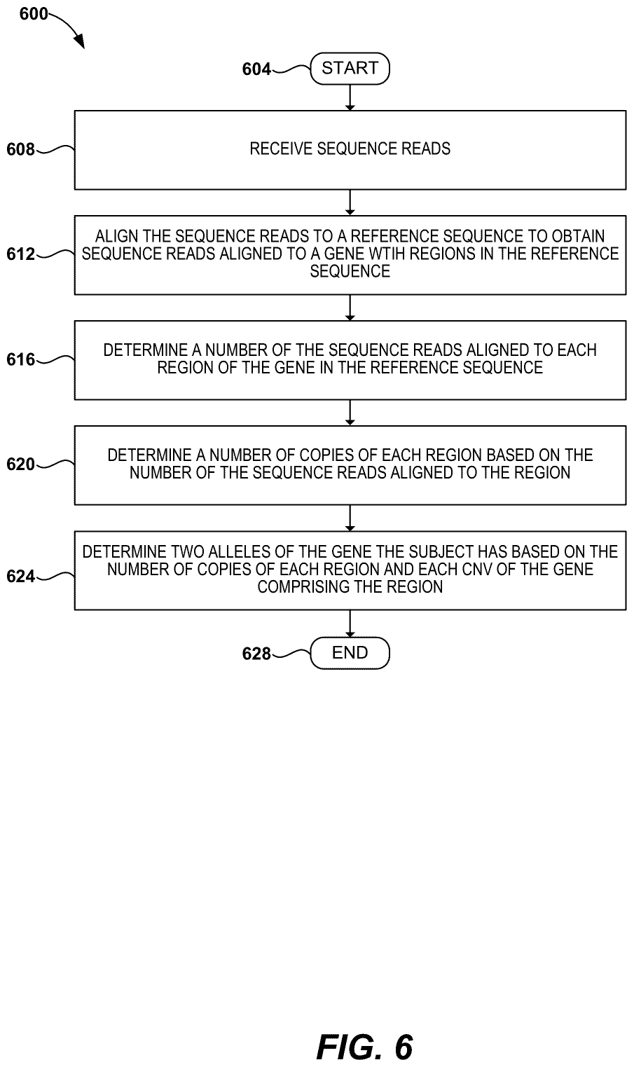

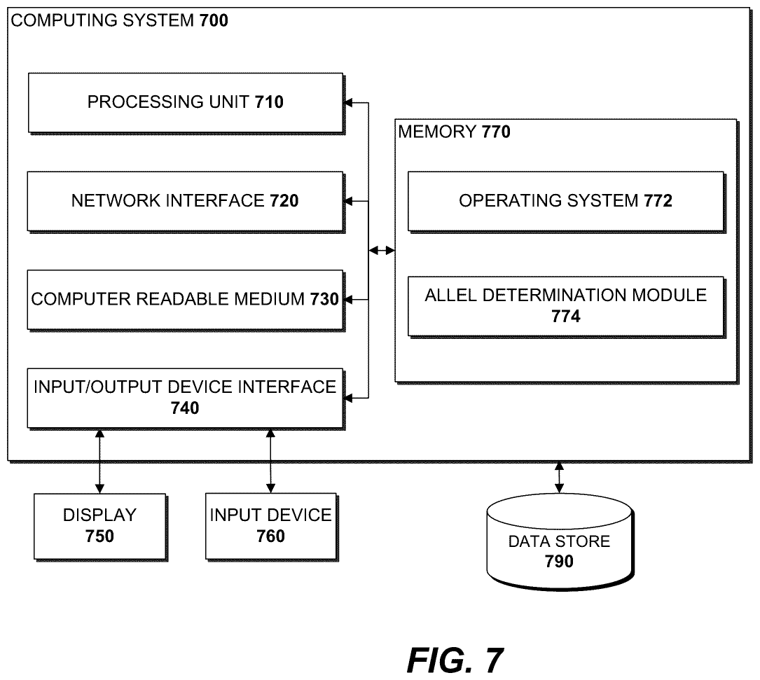

FIG. 6 is a flow diagram showing an exemplary method 600 of determining alleles of a gene. The gene can have overlapping CNVs. The method 600 may be embodied in a set of executable program instructions stored on a computer-readable medium, such as one or more disk drives, of a computing system. For example, the computing system 700 shown in FIG. 7 and described in greater detail below can execute a set of executable program instructions to implement the method 600. When the method 600 is initiated, the executable program instructions can be loaded into memory, such as RAM, and executed by one or more processors of the computing system 700. Although the method 600 is described with respect to the computing system 700 shown in FIG. 7, the description is illustrative only and is not intended to be limiting. In some embodiments, the method 600 or portions thereof may be performed serially or in parallel by multiple computing systems.

The method 600 can be efficient compared to other CNV calling methods, such as genome-wide CNV calling methods. Rediscovering the same variant in every sample using, for example, genome-wide CNV calling, can be very inefficient. In contrast, the method 600 can utilize prior knowledge of some or all possible CNVs that could exist in a given region of a gene, such as the CNVs shown in Table 1. Alternatively or additionally, the method 600 can be accurate compared to other CNV calling methods, such as genome-wide CNV calling methods. Due to the overlapping positions of CNVs, genome-wide CNV calling methods may be inaccurate, for example, when there is a mixture of signals from more than one CNV in a single sample. In contrast, the annotations generated or determined by the method 600 can be accurate. For example, method 600 can determine a subject (or the subject's sample) has an allele with V2 deletion and another allele with V4 deletion and V5 duplication illustrated in FIGS. 5A-5B and the accompanying descriptions, which is beyond the capability of genome-wide CNV calling methods. Alternatively or additionally, the method 600 can have high sensitivity. Genome-wide CNV calling methods can have low sensitivity. For example, as described with reference to FIGS. 3A-3B, genome-wide CNV calling methods would be unable to the correct calls. A genome-wide CNV calling method can determine there is duplication (CN of 3) in the first region (r1), no duplication or deletion (CN of 2) in the second region (r2), and deletion (CN of 1) in the third region (r3) in a subject (or the subject's sample). Since both regions are less than 10 kilobases in length, the difference in CN (CN of 3 or 1 in this example) from the CN of the reference (CN of 2) can be flattened by a genome-wide calling method. In contrast, the method 600 can determine the subject (or the subject's sample) has one copy of CNV V1 (which includes the first region (r1) and the second region (r2) of a gene and one copy of CNV V2 (which includes the second region (r2) and the third region (r2) of the gene described in FIGS. 3A-3B and the accompanying descriptions. In some embodiments, the method 600 may not be limited to large CNVs or regions of CNVs (e.g., 10 kb or longer) and can work with smaller CNVs or regions of CNVs (e.g., 9 kb, 8 kb, 7 kb, 6 kb, 5 kb, 4 kb, 3 kb, 2 kb, or 1 kb). Genome-wide CNV calling methods may flatten differences in CN (e.g., CN of 3 or 1) in short regions (e.g., regions that are 10 kb or shorter). The breakpoints determined using the method 600 can be, for example, precise. For example, the breakpoints determined can have single bp precisions. For example, the precision of the breakpoints determined can be in the 10s of bps or 100s of bps.

After the method 600 begins at block 604, the method 600 proceeds to block 608, where a computing system receive a plurality of sequence reads. The plurality of sequence reads can be generated from a sample. The sample can be obtained from a subject. Sequence reads can be, for example, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more base pairs in length each. For example, sequence reads are about 100 base pairs to about 1000 base pairs in length each. The sequence reads can comprise paired-end sequence reads. The sequence reads can comprise single-end sequence reads. The sequence reads can be generated by whole genome sequencing (WGS). The WGS can be clinical WGS (cWGS). The sequence reads can be generated by targeted sequencing, such as sequencing of 5, 10, 20, 30, 40, 50, 100, 200, or more genes.

The sample can comprise cells, cell-free DNA, cell-free fetal DNA, amniotic fluid, a blood sample, a biopsy sample, or a combination thereof. The sample can be obtained directly from a subject. The sample can be generated from another sample obtained from a subject. The other sample can be obtained directly from the subject or the other sample can be generated from another sample obtained from the subject. The computing system can store the plurality of sequence reads in its memory. The computing system can load the plurality of sequence reads into its memory. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA).

The method 600 proceeds from block 608 to block 612, where the computing system aligns the plurality of sequence reads to a reference sequence to obtain a plurality of aligned sequence reads comprising sequence reads aligned to a gene in the reference sequence. The gene can comprise a plurality of regions. The reference sequence can be, for example, a reference genome sequence, such as hg19 or hg38. Two copy number variants (CNVs) of a plurality of CNVs (or variants) of the gene can each comprise one or more regions of the plurality of regions. The two CNVs can differ by at least one region of the plurality of regions. Each CNV of the plurality of CNVs of the gene can comprise one or more (such as 1, 2, 3, 4, 5, 6, 7, 8, 9, 10, or more) regions of the plurality of regions. One, one or more, or each CNV of the plurality of CNVs can differ from every other CNV of the plurality of CNVs by at least one region (such as 1, 2, 3, 4, 5, 6, 7, 8, 9, 10, or more) of the plurality of regions.

The plurality of regions can comprise consecutive and/or non-overlapping regions. The number of the plurality of regions can be, or be about, 2, 3, 4, 5, 6, 7, 8, 9, 10, 15, 25, 30. For example, the plurality of regions can comprise 2 to 10 regions. FIGS. 3A-3B illustrate a gene with three regions. FIG. 4 illustrates a gene with three regions. FIGS. 5A-5B illustrate a gene with nine regions. One, one or more, or each of the plurality of regions can be, 1, 2, 3, 4, 5, 6, 7, 8, 9, 10, 15, 20, 30, 40, 50, 60, 70, 80, 90, or 100 kilobase to 100 kilobase in length. For example, one, one or more, or each of the plurality of regions can be 1 kilobase to 100 kilobase in length.

The number of the plurality of CNVs can be different in different embodiments, such as 2, 3, 4, 5, 6, 7, 8, 9, 10, 15, 20. For example, the number of the plurality of CNVs can be 2 to 10. In some embodiments, one CNV of the plurality of CNVs do not overlap with one or more (such as 1, 2, 3, 4, 5, 6, 7, 8, 9, 10 or more) other CNVs of the plurality of CNVs. Two or more (such as 2, 3, 4, 5, 6, 7, 8, 9, 10 or more) of the plurality of CNVs do not overlap or do not comprise an identical region. CNVs of a gene that do not overlap or do not comprise an identical region are non-overlapping CNVs. No CNVs of the plurality of CNVs may overlap. All CNVs of the plurality of CNVs can be non-overlapping CNVs. In some embodiments, one CNV of the plurality of CNVs overlaps with one or more (such as 1, 2, 3, 4, 5, 6, 7, 8, 9, 10, or more) other CNVs of the plurality of CNV. Two CNVs of the plurality of CNVs of the gene can overlap or can comprise an identical region of the plurality of regions. Two CNVs that overlap or comprise an identical region of the plurality of regions are overlapping CNVs.

As an example, a first region, a second region, and a third region of the plurality of regions can be consecutive and non-overlapping. A first CNV of the two CNVs can comprise the first region and the second region, not the third region (see FIG. 3A, top left panel for an illustration). A second CNV of the two CNVs can comprise the second region and the third region, not the first region (see FIG. 3A, top left panel for an illustration). As another example, a first region, a second region, and a third region of the plurality of regions are consecutive and non-overlapping. A first CNV of the two CNVs can comprise the first region, the second region, and the third region (see FIG. 4, left panel for an illustration). A second CNV of the two CNVs can comprise the second region, not the first region and the third region (see FIG. 4, left panel for an illustration). A first CNV and a second CNV of the plurality of CNVs can comprise no common region (see FIG. 5A for an illustration).

The plurality of CNVs can be predetermined, or the plurality of CNVs can be known (see FIGS. 3A-3B, 4, and 5A-5B and table 1 for illustrations). The plurality of regions can be predetermined (see FIGS. 3A-3B, 4, and 5A-5B and table 1 for illustrations). Table 1 shows the start and end positions (or approximate start and end positions) of variants and regions of genes. In some embodiments, the computing system can receive the plurality of CNVs. The computing system can determine the plurality of regions using the plurality of CNVs (see the accompanying descriptions of table 1 for illustrations). The computing system can determine the plurality of CNVs, for example, using a one-dimensional mixture of Gaussians with constrained means. The constrained means can be, for example, 0, 1, 2, 3, 4, 5, 6, 7, 8, 9, 10, or more.

The computing system can align sequence reads to the reference sequence using an aligner or an alignment method such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMER, QPalma, RazerS, REAL, cREAL, RMAP, rNA, RT Investigator, Segemehl, SeqMap, Shrec, SHRiMP, SLIDER, SOAP, SOAP2, SOAP3 and SOAP3-dp, SOCS, SSAHA and SSAHA2, Stampy, SToRM, Subread and Subjunc, Taipan, UGENE, VelociMapper, XpressAlign, and ZOOM.

The method 600 proceeds from block 612 to block 616, where the computing system determines a number of the sequence reads aligned to each region of the plurality of regions of the gene in the reference sequence. The number of the sequence reads aligned to each region of the plurality of regions of the gene can comprise a raw number or a normalized and/or GC-corrected number of the sequence reads aligned to each region of the plurality of regions of the gene. Determining the number of copies of each region of the plurality of regions can comprise determining the number of copies of each region of the plurality of regions using the number of the sequence reads aligned to the region based on a normalized and/or GC-corrected number of the sequence reads aligned to each region of the plurality of regions of the gene in the reference sequence.

The computing system can further determine the normalized number of the sequence reads aligned to each region of the plurality of regions of the gene in the reference sequence. The computing system can further determine the normalized number of the sequence reads aligned to each region of the plurality of regions of the gene in the reference sequence using (1a) a depth of the sequence reads aligned to each region of the plurality of regions of the gene in the reference sequence. The computing system can further determine the normalized number of the sequence reads aligned to each region of the plurality of regions of the gene in the reference sequence using (1b) a length of the region of the gene. The computing system can further determine the normalized number of the sequence reads aligned to each region of the plurality of regions of the gene in the reference sequence using (2a) a depth of sequence reads of the plurality of sequence reads aligned to each of a plurality of regions of the reference sequence other than a genetic locus comprising the gene. The computing system can further determine the normalized number of the sequence reads aligned to each region of the plurality of regions of the gene in the reference sequence using (2b) a length of each of the plurality of regions of the reference sequence other than the genetic locus comprising the gene. The computing system can further determine the GC corrected number of the sequence reads aligned to each region of the plurality of regions of the gene in the reference sequence from the number or the normalized number of the sequence reads aligned the region of gene in the reference sequence using a GC content of the region of the gene in the reference sequence.

The method 600 proceeds from block 616 to block 620, where the computing system determines a number of copies (or observed, estimated or determined copies) of each region of the plurality of regions based on the number of the sequence reads aligned to the region. The number of copies of each region comprises the number of copies of each region relative to a reference number of copies of the region. Such a number of copies of a region can be a change in the number of copies of the region, relative to the reference. The reference can be 2 (or 3, 4, 5, 6, 7, 8, 9, 10, or more). For example, the number of copies of the region r1 illustrated in FIG. 5B is two, and the change in the number of copies of the region r1 is zero. As another example, the number of copies of the region r4 illustrated in FIG. 5B is zero, and the change in the number of copies of the region r4, relative to a reference of two, is negative two. To determine the number of copies of each region of the plurality of regions, the computing system can determine a difference in the number of copies of each region of the plurality of regions, relative to a reference number of copies of the region, based on the number of the sequence reads aligned to the region.

The method 600 proceeds from block 620 to block 624, where the computing system determines two alleles of the gene of the subject (e.g., an allele has V2 deletion and another allele with V4 deletion and V5 duplication). The computing system can determine two alleles of the gene of the subject based on the number of copies (or the change in the number of copies) of each region of the plurality of regions and all CNVs (or each CNV) of the plurality of CNVs comprising the region. For example, the number of copies (or the change in the number of copies) of region r1 in FIG. 3B, left panel can be the number of copies (or the change in the number of copies) of CNV V1 can be used. As another example, the number of copies (or the change in the number of copies) of region r2 in FIG. 3B, left panel can be the sum of the number of copies (or the change in the number of copies) of CNV V1 and the number of copies (or the change in the number of copies) of CNV V2 can be used. As a further example, the number of copies (or the change in the number of copies) of region r3 in FIG. 3B, left panel, can be the number of copies (or the change in the number of copies) of CNV V2 can be used. See FIG. 4B and FIG. 5A and accompanying descriptions of the relationship between the number of copies (or the change in the number of copies) of a region and the number of copies (or the change in the number of copies) of each of one or more variants. Each of the two alleles of the gene of the subject can comprise one or more (such as 1, 2, 3, 4, 5, 6, 7, 8, 9, 10, or more) regions of the plurality of regions.

As an example, a first CNV of two CNVs can comprise the first region and the second region, not the third region (e.g., V1 in FIG. 3A, top left panel). A second CNV of the two CNVs can comprise the second region and the third region, not the first region (e.g., V2 in FIG. 3A, top left panel). The computing system can determine two alleles of the gene of the subject based on the number of copies of the first region, the number of copies of the second region, and the number of copies of the third region (see FIG. 3B, left panel for an illustration). As another example, a first CNV of the two CNVs can comprise the first region, the second region, and the third region (e.g., V1 in FIG. 4, left panel). A second CNV of the two CNVs can comprise the second region, not the first region and the third region (e.g., V2 in FIG. 4, left panel). The computing system can determine two alleles of the gene of the subject based on the number of copies of the first region, the number of copies of the second region, and the number of copies of the third region (see FIG. 4, left panel for an illustration). Alternatively or additionally, the computing system can determine two alleles of the gene of the subject based on the number of copies of the first region and the number of copies of the second region, not the number of copies of the third region (see FIG. 4, left panel for an illustration). The third region can be shorter or substantially shorter than the first region. The number of copies of the first region and the number of copies of the third region can be identical (e.g., region r1 and the 1 kb region in FIG. 4, left panel).

To determine the two alleles of the gene of the subject, the computing system can determine (i) the number of copies a first CNV in a first allele of the two alleles of the gene of the subject and (ii) a number of copies of a second CNV in a second allele of the two alleles of the gene of the subject such that (a) the number of copies of a region of the plurality of regions in the first CNV and not the second CNV is the number of copies of the first CNV (e.g., region r1 in FIG. 3B, left panel, region r1 in FIG. 4, left panel). Alternatively or additionally, to determine, the two alleles of the gene of the subject, the computing system can determine (i) the number of copies a first CNV in a first allele of the two alleles of the gene of the subject and (ii) a number of copies of a second CNV in a second allele of the two alleles of the gene of the subject such that (b) the number of copies of a region of the plurality of regions in the first CNV and the second CNV is the sum of the number of copies of the first CNV and the number of copies of the second CNV (e.g., region r2 in FIG. 3B, left panel, region r3 in FIG. 4, left panel). Alternatively or additionally, to determine, the two alleles of the gene of the subject, the computing system can determine (i) the number of copies a first CNV in a first allele of the two alleles of the gene of the subject and (ii) a number of copies of a second CNV in a second allele of the two alleles of the gene of the subject such that (c) the number of copies of a region of the plurality of regions in the second CNV and not the first CNV is the number of copies of the second CNV (e.g., region r3 in FIG. 3B, left panel).

The computing system can determine the two alleles of the gene of the subject using the difference in the number of copies of each region of the plurality of regions, relative to the reference number of copies of the region, and one, one or more, or each CNV of the plurality of CNVs comprising the region. For example, as illustrated in FIG. 3B, left panel, and the accompanying descriptions, the difference in the number of (observed) copies of region r1, relative to the reference number of two; the difference in the number of (observed) copies of region r2, relative to the reference number of two; and the difference in the number of (observed) copies of region r3, relative to the reference number of two, can be used to determine the two alleles of the gene of the subject.

The two alleles of the subject can be identical. The two alleles of the subject can be different. A first allele of the two alleles of the subject can comprise a duplication (e.g., having two copies) of a CNV of the plurality of CNVs. A first allele of the two alleles can comprise a deletion (e.g., having zero copy) of a CNV of the plurality of CNVs. For example, one allele described with reference to FIG. 5B has a deletion of the CNV V4 and a duplication of the CNV V5. A first allele of the two alleles can comprise or be one copy of a CNV of the plurality of CNVs. A second allele of the two alleles can comprise a duplication (e.g., having two copies) of a CNV of the plurality of CNVs. A second allele of the two alleles can comprise a deletion (e.g., having zero copy) of a CNV of the plurality of CNVs. A second allele of the two alleles can comprise or be one copy of a CNV of the plurality of CNVs.

The computing system can create a file or a report representing or comprising alleles of the gene of the subject and/or the one or more regions of the plurality of regions in each of the two alleles. The computing system can generate a user interface (UI) comprising a UI element representing or comprising alleles of the gene of the subject and/or the one or more regions of the plurality of regions in each of the two alleles. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion).

The method 600 ends at block 628.

### Execution Environment

FIG. 7 depicts a general architecture of an example computing device 700 configured to execute the processes and implement the features described hereinf. The general architecture of the computing device 700 depicted in FIG. 7 includes an arrangement of computer hardware and software components. The computing device 700 may include many more (or fewer) elements than those shown in FIG. 7. It is not necessary, however, that all of these generally conventional elements be shown in order to provide an enabling disclosure. As illustrated, the computing device 700 includes a processing unit710, a network interface720, a computer readable medium drive730, an input/output device interface740, a display 750, and an input device760, all of which may communicate with one another by way of a communication bus. The network interface720 may provide connectivity to one or more networks or computing systems. The processing unit 710 may thus receive information and instructions from other computing systems or services via a network. The processing unit710 may also communicate to and from memory 770 and further provide output information for an optional display 750 via the input/output device interface 740. The input/output device interface 740 may also accept input from the optional input device 760, such as a keyboard, mouse, digital pen, microphone, touch screen, gesture recognition system, voice recognition system, gamepad, accelerometer, gyroscope, or other input device.

The memory 770 may contain computer program instructions (grouped as modules or components in some embodiments) that the processing unit 710 executes in order to implement one or more embodiments. The memory770 generally includes RAM, ROM and/or other persistent, auxiliary or non-transitory computer-readable media. The memory770 may store an operating system772 that provides computer program instructions for use by the processing unit 710 in the general administration and operation of the computing device700. The memory770 may further include computer program instructions and other information for implementing aspects of the present disclosure.

For example, in one embodiment, the memory770 includes an allele determination module 774 for determining (or calling) alleles of a subject, such as the method 600 described with reference to FIG. 6. In addition, memory 770 may include or communicate with the data store 790 and/or one or more other data stores that that store the input and/or output of the method 600, such as the sequence reads, regions of a gene, copy number variants of a gene, the number of copies of a region of a gene, and the alleles of the gene the subject has.

### Additional Considerations

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein In which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1. A method for determining alleles of a gene of a subject comprising:
under control of a hardware processor:
receiving a plurality of sequence reads generated from a sample obtained from a subject;
aligning the plurality of sequence reads to a reference genome sequence to obtain a plurality of aligned sequence reads comprising sequence reads aligned to a gene in the reference genome sequence, wherein the gene comprises a plurality of regions, wherein two copy number variants (CNVs) of a plurality of CNVs of the gene each comprises one or more regions of the plurality of regions and differ by at least one region of the plurality of regions;
determining a number of the sequence reads aligned to each region of the plurality of regions of the gene in the reference genome sequence;
determining a number of copies of each region of the plurality of regions based on the number of the sequence reads aligned to the region; and
determining two alleles of the gene of the subject, each comprising one or more regions of the plurality of regions, based on the number of copies of each region of the plurality of regions and all CNVs of the plurality of CNVs comprising the region.

2. The method of claim 1, wherein the plurality of regions comprises a plurality of consecutive regions.

3.-5. (canceled)

6. The method of claim 1, wherein no CNVs of the plurality of CNVs overlap.

7. The method of claim 1, wherein the two CNVs of the plurality of CNVs of the gene do not overlap.

8. The method of claim 1, wherein the two CNVs of the plurality of CNVs of the gene overlap.

9. The method of claim 1, wherein the two CNVs of the plurality of CNVs of the gene comprise an identical region of the plurality of regions.

10. The method of claim 1, wherein each CNV of the plurality of CNVs of the gene comprises one or more regions of the plurality of regions, and wherein each CNV of the plurality of CNVs differ from every other CNV of the plurality of CNVs by at least one region of the plurality of regions.

11. The method of claim 1, wherein a first region, a second region, and a third region of the plurality of regions are consecutive and non-overlapping, wherein a first CNV of the two CNVs comprises the first region and the second region, not the third region, and wherein a second CNV of the two CNVs comprises the second region and the third region, not the first region.

12. The method of claim 1, wherein a first region, a second region, and a third region of the plurality of regions are consecutive and non-overlapping, wherein a first CNV of the two CNVs comprises the first region, the second region, and the third region, and wherein a second CNV of the two CNVs comprises the second region, not the first region and the third region.

13. (canceled)

14. The method of claim 1, wherein a first CNV and a second CNV of the plurality of CNVs comprise no common region.

15. The method of claim 1, wherein the number of the sequence reads aligned to each region of the plurality of regions of the gene comprises a raw number or a normalized and/or GC-corrected number of the sequence reads aligned to each region of the plurality of regions of the gene.

16. The method of claim 1, wherein determining the number of copies of each region of the plurality of regions comprises: determining the number of copies of each region of the plurality of regions using the number of the sequence reads aligned to the region based on a normalized and/or GC-corrected number of the sequence reads aligned to each region of the plurality of regions of the gene in the reference genome sequence.

17. (canceled)

18. (canceled)

19. The method of claim 1, wherein the number of copies of each region comprises the number of copies of each region relative to a reference number of copies of the region, optionally wherein the reference number of copies of the region is 2.

20. The method of claim 1,
wherein determining the number of copies of each region of the plurality of regions comprises: determining a difference in the number of copies of each region of the plurality of regions, relative to a reference number of copies of the region, based on the number of the sequence reads aligned to the region, and
wherein determining the two alleles of the gene of the subject comprises: determining the two alleles of the gene of the subject using the difference in the number of copies of each region of the plurality of regions, relative to the reference number of copies of the region, and all CNVs of the plurality of CNVs comprising the region.

21. The method of claim 1, wherein a first allele of the two alleles comprises a duplication of a CNV of the plurality of CNVs and/or a deletion of a CNV of the plurality of CNVs.

22. The method of claim 1, wherein a first allele of the two alleles comprises one copy of a CNV of the plurality of CNVs.

23. (canceled)

24. (canceled)

25. The method of claim 1, wherein determining the two alleles of the gene of the subject comprises: determining (i) a number of copies a first CNV in a first allele of the two alleles of the gene of the subject and (ii) a number of copies of a second CNV in a second allele of the two alleles of the gene of the subject such that (a) the number of copies of a region of the plurality of regions in the first CNV and not the second CNV is the number of copies of the first CNV, (b) the number of copies of a region of the plurality of regions in the first CNV and the second CNV is the sum of the number of copies of the first CNV and the number of copies of the second CNV, and/or (c) the number of copies of a region of the plurality of regions in the second CNV and not the first CNV is the number of copies of the second CNV.

26. (canceled)

27. The method of claim 1, further comprising:
receiving the plurality of CNVs; and
determining the plurality of regions using the plurality of CNVs, optionally wherein receiving the plurality of CNVs comprises determining the plurality of CNVs.

28. The method of claim 1, further comprising: creating a file or a report and/or generating a user interface (UI) comprising a UI element representing or comprising alleles of the gene of the subject and/or the one or more regions of the plurality of regions in each of the two alleles.

29.-32. (canceled)

33. A system for determining alleles of a gene of a subject comprising:
non-transitory memory configured to store executable instructions, a plurality of regions of a gene, and a plurality of copy number variants (CNVs) of the gene, wherein two CNVs of the plurality of CNVs of the gene each comprises one or more regions of the plurality of regions and differ by at least one region of the plurality of regions; and
a hardware processor in communication with the non-transitory memory, the hardware processor programmed by the executable instructions to perform:
receiving a plurality of sequence reads generated from a sample obtained from a subject;
aligning the plurality of sequence reads to a reference sequence to obtain a plurality of aligned sequence reads comprising sequence reads aligned to the gene in the reference genome sequence;
determining a number of copies of each region of the plurality of regions based on a number of the sequence reads aligned to the region; and
determining two alleles of the gene of the subject, each comprising one or more regions of the plurality of regions, based on the number of copies of each region of the plurality of regions and all CNVs of the plurality of CNVs comprising the region.

34.-66. (canceled)
